(19) United States
(12) Patent Application Publication
(10) Pub. No.: US 2003/0199486 A1
(43) Pub. Date: Oct. 23, 2003

(54) Ester and ether derivatives of 4-hydroxy-19-nortestosterone and a method for the regulation of athletic function in humans

(75) Inventor: Sal Abraham, Dunmore, PA (US)

Correspondence Address: SAL ABRAHAM, 1304 ELECTRIC ST., DUNMORE, PA, 18509, US

(73) Assignee: Nutri Sport Pharmacal, Inc., Franklin, NJ (US)

(21) Appl. No.: 10/064,415

(22) Filed: Jul. 11, 2002

Related U.S. Application Data
(63) Continuation-in-part of application No. 10/063,415, filed on Apr. 22, 2002, now Pat. No. 6,586,417.
(63) Continuation-in-part of application No. 10/064,027, filed on Jun. 4, 2002.
(63) Continuation-in-part of application No. 10/064,277, filed on Jun. 27, 2002.

(51) Int. Cl. A61K 31/58; A61K 31/56

(52) U.S. Cl. 514/172; 514/178

(57) ABSTRACT

This invention relates to a method of administering an effective extended release amount of at least one of the ester or ether derivatives of 4-hydroxy-19-nortestosterone for the regulation of athletic function in humans as a dietary supplement.

This is a continuation in part of Ser. No. 10/063,415, Filed on Apr. 22, 2002, (pending), Ser. No. 10/064,027, Filed on Jun. 4, 2002, (pending), and Ser. No. 10/064,277, Filed on Jun. 27, 2002, (pending).

### BACKGROUND OF INVENTION

This invention relates to the ester and ether derivatives of 4-hydroxy-19-nortestosterone as a means of regulating athletic function in humans as a dietary supplement. In men, the normal balance of sex steroids is characterized by a greater amount of androgens over estrogens. When estrogen exceeds androgen levels in males a cascade of detrimental effects can take place for instance, decreased sperm count, low free testosterone levels, decreased muscle mass and decreased strength. When androgens are manipulated to exceed estrogen above the normal androgen to estrogen ratio the exact opposite takes place with an emphasis on the promotion of lean body mass and strength to aid in enhancing physical performance.

Serious and/or professional athletes are known to utilize intramuscular injection and/or peroral administration of pharmaceutical androgens for the promotion of muscle mass and athletic performance. Various testosterone esters in oil depot form have been utilized as intramuscular injection. These weekly injections of synthetic testosterone cause supraphysiological surges in androgen levels which then leave the body deficient in androgens until the next injection. These supraphysiological levels testosterones readily convert to estrogen and dihydrotestosterone (DHT) which can lead to side effects such as gynocomastia and benign prostrate hypertrophy (BPH). Another negative side effect is the possible down regulation of the hypothalamic pituitary axis resulting in loss of natural testosterone production.

The oral route of synthetic androgen administration consists of testosterone derivatives that are 17 alpha-alkylated for enhanced oral bioavailability (i.e. Methyltestosterone). This alkylation allows the steroids to withstand the 17 beta-hydroxyl oxidation in the liver. This appears to alleviate the dosing and blood hormone problems as compared to injections but it can cause undue stress to the and increase the possibility of hepatotoxicity.

U.S. Pat. No. 6,242,436 to William Llewellyn, relates a method of using the oral precursor hormones 5alpha-androstanediol or 5alpha-androstanedione as a means of increasing dihydrotestosterone levels in humans. Dihydrotestosterone (DHT) is a more potent form of testosterone, shown to be roughly three to four times more active in the human body in comparison. This hormone represents an improvement, since 5alpha-androstanediol or 5alpha-androstanedione are natural, non-toxic, and quickly metabolized to active form after oral administration and unable to be aromatized into estrogens due to their structure. The compound suggested in this patent does offer advantages over standard therapies in that the compound in question avoids a direct path of estrogen conversion and provides a less toxic peroral route of administration. However, dihydrotestosterone (DHT) is not the most ideal form of testosterone due to it's causative relationship with benign prostrate hypertrophy (BPH) and other androgenic side effects.

U.S. Pat. No. 2,762,818 to Levy, et al. discloses a method for the synthetic manufacturing of various esters of 4-hydroxytestosterone and discloses a medical use for the treatment of various states of androgen deficiency due to 4-hydroxytestosterone's anabolic and androgenic properties. The medical treatment referred to in this patent deals with the pharmaceutical application of 4-hydroxytestosterone for specific disease states. As with most androgens, 4-hydroxytestosterone is noted to increase the retention of nitrogen in metabolic processes. The majority of the patent then discloses eleven different methods for the synthesis of various esters of 4-hydroxytestosterone.

U.S. Pat. No. 6,011,027 to Patrick Arnold, relates a method of using of using the oral precursor hormone 19-nor-4-androstenediol as a means of increasing 19-nortestosterone levels in humans. This hormone represents an improvement in standard therapies, since 19-nor-4-androstenediol converts into 19-nortestosterone thereby imparting the same anabolic effect as testosterone with much less androgenic activity. The possibility that this compound will convert to an estrogen over an androgen is chemically unlikely. The end product 19-nortestosterone also exhibits much less aromatization than testosterone. The compound suggested in this patent does offer advantages over standard therapies in that the compound in question avoids a direct path of estrogen conversion and provides a less toxic peroral route of administration. The target hormone of replacement may still be less than ideal due to it's poor oral absorption and bioavailability.

U.S. patent application Ser. Nos. 10/063,415, 10/064027, and 10/064,277 also by Sal Abraham, discloses a method of effectively increasing natural or endogenous testosterone, 4-hydroxytestosterone, and 4-hydroxy-19-nortestosterone levels in humans by the oral administration of the ester and ether derivatives of 4-hydroxy-4-androstenedione, 4-hydroxytestosterone, and 4-hydroxy-19-norandrosterone. U.S. patent application Ser. No. 10/063,415 demonstrates 4-hydroxy-4-androstenedione's ability lower estrogen and thus stimulate testosterone for the promotion of the variables associated with athletic function thereby enhancing physical performance. U.S. patent application Ser. No. 10/064,027, discloses 4-hydroxytestosterone as an natural anabolic/androgenic metabolite of 4-hydroxy-4-androstenedione, which is an analog of the naturally occurring dietary supplement 4-androstenedione. U.S. patent application Ser. No. 10/064,277 discloses a method for the use of ester and ether pro-steroids or precursors such as 4-hydroxy-19-norandrosterone to increase the level of 4-hydroxy-19-nortestosterone. To the best of my knowledge, the use of 4-hydroxy-4-androstenedione and 4-hydroxytestosterone for the regulation of athletic function as a dietary supplement has never been disclosed. Also to the best of my knowledge, the use of 4-hydroxy-19-norandrosterone to increase the level of 4-hydroxy-19-nortestosterone has never been disclosed in the literature. The fact that 4-hydroxytestosterone is naturally made in the body during the metabolism of 4-hydroxy-4-androstenedione is not widely known and is justification for its use as a dietary supplement since 4-hydroxy-4-androstenedione is found in nature. For this same reason, 4-hydroxy-4-androstenedione can also be sold as a dietary supplement, although this to is not widely known or understood. The esters and ethers of 4-hydroxy-4-androstenedione impart an anabolic/androgenic effect and represent a novel and unobvious improvement in the regulation of athletic function. 4-hydroxytestosterone and 4-hydroxy-19-norandrosterone also exhibits unique characteristics over other androgenic/anabolic hormones wherein they both have the ability to impart a mild androgenic effect with a potent anabolic effect without causing hepatotoxicity, DHT accumulation, supraphysiological surges in androgen blood concentrations, and down regulation of the hypothalamic pituitary axis. However, direct supplementation of 4-hydroxy-19-nortestosterone represents still a further improvement over standard anabolic/androgenic therapies due to greater oral bioavailability and also represents a novel and unobvious improvement in athletic function as a dietary supplement.

### SUMMARY OF INVENTION

The use of illicit synthetic exogenous testosterone or nortestosterone for the promotion of muscle mass and work performance is well known throughout athletic circles. However these therapies, besides being illegal, result in the aromatization of the target hormone and put undue stress upon the liver. Dietary supplement manufactures have attempted to correct these problems by the use of pro-hormones for the promotion of physical performance. For instance, U.S. Pat. Nos. 6,011,027 and 6,242,436 attempt to correct some of these problems by the peroral administration of androgen precursors. Now while the active androgen precursor is unable to aromatize, the total accumulation of end product is minimal due to the poor oral absorption and bioavailability of U.S. Pat. No. 6,011,027 while the target hormone of U.S. Pat. No. 6,242,436 is directly converted to dihydrotestosterone (DHT). The problem of the present invention is to provide a compound that gradually increases 4-hydroxy-19-nortestosterone levels for the promotion of fat free mass and athletic performance as a dietary supplement without causing DHT accumulation, hepatotoxicity, supraphysiological surges in androgen blood concentrations, and down regulation of the hypothalamic pituitary axis. According to the invention these problems are solved by the use of 4-hydroxy-19-nortestosterone, which is an analog of the naturally occurring hormone 19-nortestosterone. The use of this compound involves a method of imparting an anabolic effect in humans for the promotion lean body mass and physical performance with none of the previous mentioned negative side effects. 4-hydroxy-19-nortestosterone is a metabolite of the naturally occurring hormone 19-nortestosterone and therefore can be sold as a dietary supplement. To the best of my knowledge, 4-hydroxy-19-nortestosterone has also never been commercially available in the United States as a supplement or a drug. The ester and ether analogs of this compound can also be sold as dietary supplements as long as the ester and ether additions are utilized for increasing oral bioavailability. The addition of various ethers to 4-hydroxy-19-nortestosterone are intended to increase oral bioavailability and have not been previously demonstrated in the literature. The use of a oral extended release version of 4-hydroxy-19-nortestosterone as dietary supplement represents a novel and unobvious improvement in athletic function due to its not widely known metabolic functions and chemical classification which in turn allows this compound to be sold as a dietary supplement and not require it be sold as a pharmaceutical drug.

### DETAILED DESCRIPTION

The chemical term 4-hydroxy-19-nortestosterone refers to two isomers: 4alpha,17beta-Dihydroxyestr-4-en-3-one and 4beta,17beta-Dihydroxyestr-4-en-3-one. This invention concerns both isomer forms of 4-hydroxy-19-nortestosterone. 4-hydroxy-19-nortestosterone is an metabolite of the naturally occurring hormone 19-nortestosterone. Viable chemical esters of this compound include methyl, ethyl, propyl, butyl, and cyclohexyl carbonate as well as, acetate, heptanoate, decanoate, hemisuccinate, and benzoate. Viable chemical ethers include tetrahydropyranyl (THP), cyclopentyl (CPT), cyclohexyl and tetrahydrofuranyl (THF) ethers. This invention concerns the esters and ethers of the two isomer forms of 4alpha,17beta-Dihydroxyestr-4-en3-one and 4beta,17beta-Dihydroxyestr-4-en-3-one. The previous examples of various esters and ethers are presented by way of illustration only. It should be understood that this invention is not construed as limited in scope by the details contained therein, as it is apparent to those skilled in the art that modifications in materials and methods can be made without deviating from the scope of the invention.

The base compound of 4-hydroxy-19-nortestosterone is 19-nortestosterone which is chemically close to testosterone. Recent studies by Le Bizec, B. et al. Steroids. February 2002;67(2):105-10 demonstrates the endogenous production of 19-nortestosterone in man (i.e.. 2 ng/ml) 19-nortestosterone is chemically close to testosterone. This chemical difference imparts a noticeable differentiation between these two hormones. Testosterone is considered an androgenic/anabolic hormone while 19-nortestosterone is considered primarily anabolic, since 19-nortestosterone is missing the axial methyl group C19 (carbon 19) stemming off of C10 (carbon 10). 19-nortestosterone posses approximately the same anabolic effect as testosterone without the androgenic or male virilization effects (facial hair growth, body hair growth, male pattern baldness, and lowering of voice pitch). Testosterone is the major naturally occurring androgenic hormone produced by the interstitial (Leydig) cells of the testes in response to stimulation by the luteinizing hormone of the anterior pituitary. It regulates gonadotropic secretion and wolffian duct differentiation, and stimulates skeletal muscle. It is also responsible for other male characteristics and spermatogenesis after its conversion to dihydrotestosterone.

U.S. Pat. No. 2,999,870 to Bruno Camerino, relates a method for the chemical manufacturing of 4-hydroxy-3-keto-4-androstenes. 4-hydroxytestosterone and 4-hydroxy-19-nortestosterone are claimed with various 17-alpha alkylation processing techniques. This patent claims no functions or uses other than the chemical design or processing of these compounds and represents the initial introduction of 4-hydroxy-3-keto-4-androstenes. There are nine processing examples of 4-hydroxy-17alpha-methyl-testosterone and 4-hydroxy-17-alpha-methyl-19-nortestosterone. This alkylation allows for enhanced oral bioavailability and for the steroids to withstand the 17 beta-hydroxyl oxidation in the liver, although it can cause undue stress to the liver and increase the possibility of hepatotoxicity.

U.S. Pat. No. 3,020,295 to Bruno Camerino, relates a therapeutic 4-hydroxy-19-nortestosterone-17-cyclopentyl-propionate and its process of preparation and its application. The addition of cyclopentyl-propionate to 4-hydroxy-19-nortestosterone was intended to prolong activity therefore causing an extended myotrophic action. Results indicated that 4-hydroxy-19-nortestosterone-17-cyclopentyl-propionate exhibited a higher myotrophic activity than that of testosterone-cyclopentyl-propionate on the 7th and 28th day. Androgenicity was lower than testosterone-cyclopentyl-propionate which indicated a much more favorable therapeutic index. Just 50 mg. injected once weekly demonstrated a good anabolic effect with no signs of toxicity, no increases in blood pressure, and no sodium retention.

U.S. Pat. No. 3,060,201 to Bruno Camerino, relates a method for chemical manufacturing of 4-hydroxy-17alpha-methyl-3-keto-delta4-steroids of androstane and 19-nor-androstanes series and esters thereof. There are ten examples of various esters of 4-hydroxy-17alpha-methyl-testosterone, one example of 4,11beta-dihydroxy-17-alpha-methyltestosterone, and one example of 4-hydroxy-17alpha-methyl-19-nortestosterone. This patent discusses various medical uses associated with the retention of muscle and bone mass. It then discloses 4-hydroxy-17alpha-methyl-testosterone is devoid of progestational and sodium retaining activity. The 4-hydroxy-17alpha-methyl-19-nortestosterone is also devoid of progestational activity but also posses anti-estrogenic effects. 4-hydroxy-17alpha-methyl-19-nortestosterone myotrophic activity is noted to be twenty seven times higher than 17-alpha-methyltestosterone.

4-hydroxy-19-nortestosterone is an natural anabolic/androgenic metabolite of 4-hydroxy-19-norandrostenedione, which is an analog of the naturally occurring dietary supplement 19-norandrostenedione. To the best of my knowledge, the direct use of 4-hydroxy-19-nortestosterone for the regulation of athletic function as a dietary supplement has never been disclosed. The fact that 4-hydroxy-19-nortestosterone is naturally made in the body during the metabolism of 4-hydroxy-19-norandrostenedione is not widely known and is justification for its use as a dietary supplement. 4-hydroxy-19-nortestosterone (4,17beta-Dihydroxyestr-4-en-3-one) or Oxabolone is a naturally occurring anabolic/androgenic 4-hydroxylated form of 19-nortestosterone. Steroid hydroxylation at C4 (carbon 4) has been demonstrated to occur in nature by Ding, X . et al. Arch. Biochem. Biophys. 315, 454-459. This is reaction is important in order to justify the metabolic pathway of 19-nortestosterone to 4-hydroxy-19-nortestosterone. Previous research by Waxman, D J. et al. Arch Biochem Biophys October 1991;290(1):160-6 has demonstrated that individual human cytochrome P450 enzymes can hydroxylate endogenous steroid hormones with a high degree of stereospecificity and regioselectivity and that some, but not all of the human cytochromes exhibit metabolite profiles similar to their rodent counterparts.

Research by Raeside, J I. et al. J Steroid Biochem Mol Biol July 1992;42(6):637-41 further supports a 19-norandrostane steroid C4 hydroxylation pathway. Both 11 beta and 6 beta-hydroxylated derivatives of 19-Norandrostenedione were found in porcine Leydig cells and a third product (11-oxo-19-Nor androstenedione) was tentatively identified. The profile of radioactive metabolites from [3H]19-Nortestosterone also favors the view of a capacity for hydroxylation of 19-norandrogens by porcine Leydig cells. Clouet, A S. et al. Analyst December 1998;123(12):2489-92 has demonstrated in vivo metabolism in calf hepatocyte cultures of 19-nortestosterone. 19-Norepitestosterone, the main in vivo metabolite, was identified in vitro. However, the main in vitro metabolites were mostly in an oxidized form (4-estrene-3,17-dione, hydroxy-4-estrene-3,17-dione).

The esters and ethers of 4-hydroxy-4-androstenedione impart an anabolic/androgenic effect and represent a novel and unobvious improvement in the regulation of athletic function. 4-hydroxytestosterone also exhibits unique characteristics over other androgenic/anabolic hormones wherein 4-hydroxytestosterone has the ability to impart a mild androgenic effect with a potent anabolic effect without causing hepatotoxicity, DHT accumulation, supraphysiological surges in androgen blood concentrations, and down regulation of the hypothalamic pituitary testicular axis (HPTA). However, 4-hydroxy-19-nortestosterone represents an improvement in previous 19-nor androgen hormone therapies due to no progestational activity, imparting anti-estrogenic effects, and possessing a very high anabolic to androgenic ratio. 19-nor androgens are know to have progestational activity and thus send negative feedback to the hypothalamic pituitary testicular axis (HPTA) causing a cessation of natural testosterone production. 19-nor androgens are also known to have less conversion into estrogen than testosterone but may still cause elevated estrogen levels. 4-hydroxy-19-nortestosterone possess no progestational activity and therefore should not negatively effect the hypothalamic pituitary testicular axis (HPTA). 4-hydroxy-19-nortestosterone cannot directly convert into estrogen and coupled with the anti-estrogenic effect should actually decrease circulating estrogen levels. The administration of an effective extended release amount of the esters and/or ethers of 4-hydroxy-19-nortestosterone increases the anabolic/androgenic hormone 4-hydroxy-19-nortestosterone and thus represents a novel and unobvious improvement in in the regulation of athletic function as a dietary supplement. 4-hydroxy-19-nortestosterone exhibits further improvements over other anabolic/androgenic hormones wherein its ability to impart a mild androgenic effect with a potent anabolic effect without causing hepatotoxicity, supraphysiological surges in androgen blood concentrations, and down regulation of the hypothalamic pituitary testicular axis (HPTA). 4-hydroxy-19-nortestosterone represents still a further improvement over standard anabolic/androgenic therapies and also represents a novel and unobvious improvement in athletic function as a dietary supplement.

Animal research performed by Gardi R, Falconi G, Pedrali C, Vitali R, Ercoli A (Steroids) May 19, 1972:5 639-47 demonstrates the androgenic and myogenic activity of orally administered ether modified alkyl androstan-17beta-yl mixed acetyls. The chemical addition of ethers and esters are designed to enhance oral absorption and bioavailability. The chemical addition of various ethers to the hydroxyl portion of the molecule, such as tetrahydropyranyl, tetrahydrofuranyl, cyclopentyl, or cyclohexyl ether, enables the compound to become lipophilic or fat soluble. This lipophilicity allows the compound to be absorbed via the lymphatic system and bypass the first-pass through the liver allowing more of the active compound to enter the bloodstream with no risk of hepatotoxicity. In order to increase the oral anabolic effect we have developed an extended release profile by adding matrix binders such as hydroxy propyl methyl cellulose, carnuba wax, and ethyl cellulose thereby prolonging the dissolution rate of the active ingredient into the lymphatic system. According to The Merck Manual of Diagnosis and Therapy, Section 22. Clinical Pharmacology, Chapter 298. Drug Input And Disposition, these products must disintegrate and dissolve before absorption can occur. Disintegration greatly increases the drug's surface area in contact with GI fluids, thereby promoting drug dissolution and absorption. Disintegrants and other excipients (eg, diluents, lubricants, surfactants, binders, dispersants) are often added during manufacture to facilitate these processes.

Increases in lean body mass, strength, and work performance are predominately associated with the amount of free or active testosterone available in the body. These increases contribute to the regulation of athletic function and thus lead to enhanced physical performance. The ester and ether derivatives of 4-hydroxy-19-nortestosterone have been shown to impart an anabolic/androgenic effect with none of previously mentioned negative side effects associated with increases in androgen production. Thus the said compound can be given to humans either in conjunction with or without a high protein diet (1.25 to 1.8 grams protein/kilogram of body weight) and proper anaerobic training program in order to increase the variables associated with athletic function for the purpose of enhancing physical performance. Therefore this compound represents an improvement in standard dietary androgen supplementation for the regulation of athletic performance.

After an extensive review of the scientific literature regarding the anabolic/androgenic activity and the naturally occurring formation of 4-hydroxy-19-nortestosterone, it then became the focus of this invention that the esters and ethers of 4-hydroxy-19-nortestosterone could be administrated perorally as an effective means of enhancing physical performance in humans as a dietary supplement without causing androgen related side effects. The oral daily doses can be between 10 to 2000 mg., but preferably 50 to 600 mg. The preferred daily dosing schedule should be divided into 3 sub dose applications per day in order maintain adequate blood hormone concentrations. In addition to peroral use, the esters and ethers of 4-hydroxy-19-nortestosterone can be effectively administered by several other routes including transdermal, sublingual, and intranasal.

1. A method of enhancing physical performance in humans by the administration of an effective amount of at least one of the ester or ether derivatives of 4-hydroxy-19-nortestosterone.

2. A method of claim 1, wherein said compound is 4alpha,17beta-Dihydroxyestr-4-en-3-one.

3. A method of claim 1, wherein said compound is 4beta,17beta-Dihydroxyestr-4-en-3-one.

4. A method of claim 1, wherein said ester derivatives include acetate, heptanoate, decanoate, hemisuccinate, benzoate and propionate.

5. A method of claim 1, wherein said carbonate ester derivatives include methyl, ethyl, propyl, butyl, and cyclohexyl.

6. A method of claim 1, wherein said ether derivatives include tetrahydropyranyl, tetrahydrofuranyl, cyclopentyl, and cyclohexyl.

7. A method of claim 1, wherein said administration is peroral.

8. A method of claim 1, wherein said administration is selected from the group consisting of transdermal, sublingual, and intranasal.

9. A method of claim 1, wherein said effective amount is a daily dosage of 10 mg to 2000 mg.
